(19) United States
(12) Patent Application Publication
(10) Pub. No.: US 2026/0210932 A1
(43) Pub. Date: Jul. 23, 2026

(54) Detection-While-Drilling Device for Composite Rock Stratum Structures and Intelligent Identification Method

(71) Applicants: CHINA UNIVERSITY OF MINING AND TECHNOLOGY, Jiangsu (CN); XUZHOU UNIVERSITY OF TECHNOLOGY, Jiangsu (CN); JIANGSU UNIVERSITY OF TECHNOLOGY, Jiangsu (CN)

(72) Inventors: Zhengzheng XIE, Jiangsu (CN); Feng GUO, Jiangsu (CN); Nong ZHANG, Jiangsu (CN); Qun WEI, Jiangsu (CN)

(21) Appl. No.: 18/874,256

(22) PCT Filed: Jan. 25, 2024
(86) PCT No.: PCT/CN2024/073922

§ 371 Date: Dec. 12, 2024

(30) Foreign Application Priority Data
Jul. 12, 2023 (CN) 202310852726.7

(51) Int. Cl. G01N 33/24; G01N 1/04

(52) U.S. Cl. CPC G01N 33/24; G01N 1/04

(57) ABSTRACT

The present invention discloses a detection-while-drilling device for composite rock stratum structures and an intelligent identification method. The detection-while-drilling device comprises an equipment base, a vertically-arranged supporting rod is fixedly connected to the middle of the equipment base, a drilling hydraulic oil cylinder is fixedly connected to one side of the bottom of the supporting rod, a piston end of the hydraulic oil cylinder is fixedly connected with a self-adaptive data acquisition-while-drilling module and is slidably connected with the supporting rod, a sample table is arranged above the self-adaptive data acquisition-while-drilling module and is fixedly connected with the supporting rod, a top end of the supporting rod is fixedly connected with a top limiting system, a rock gripper is arranged between the sample table and the top limiting system; and the self-adaptive data acquisition-while-drilling module, the data acquisition device and a data analysis terminal are electrically connected. The present invention restores the on-site real mechanical environment of a coal mine, multiple parameters are obtained by means of the self-adaptive data acquisition-while-drilling module, the performance parameters of composite rock strata are represented with a machine learning method, intelligent identification of rock lithology is achieved, and support can be provided for intelligent control of the surrounding rocks of coalmine roadways.

### TECHNICAL FIELD

The present invention belongs to the technical field of surrounding rock sensing during roadway excavation in coal mines, and particularly relates to a detection-while-drilling device for composite rock stratum structures and an intelligent identification method.

### BACKGROUND ART

Coal seam roadways may have significant fluctuations owing to geological conditions, and the simple support of long roadways often leads to excessive or inadequate support strength, resulting in waste of support cost and reduction of roadway maintenance and control effect. Meanwhile, under the influence of construction of intelligent stope faces, the problem of mining-excavation relay becomes more and more prominent. Therefore, the transparency and visualization of the roof rock mass structures of coal roadways has become a key issue for rapid and intelligent excavation of coal roadways.

Borehole imaging and micro-seismic monitoring, as mainstream techniques for detecting the roof structures of coal roadways at present, can't realize real-time detection of the roof structures owing to the size of integrated equipment and the construction efficiency, and can't meet the specific requirements for rapid excavation of coal roadways. As a simple and convenient detection method, detection-while-drilling was originally used in the fields of petroleum and large-scale geological exploration. In recent years, detection-while-drilling has been introduced gradually into the coal mine field with the development of intelligent excavation. A mapping relationship between different drilling parameters and the rock mass is established mainly by acquiring parameters such as torque, rotation speed and propulsion force in real time during drilling with a drilling machine.

The device and method for testing the structural characteristics of a rock mass while drilling proposed in CN114000866A realize acquisition of drilling process parameters by means of a rock mass fixing device and a drilling part. However, the device can't set a confining pressure and can't realize multi-source acquisition of drilling parameters, therefore it can't achieve realistic simulation of actual engineering conditions. The dynamic detection and evaluation method for impact risk based on response parameters while drilling proposed in CN112145151A is mainly used to reasonably analyze different parameters during drilling, so as to simulate the stress environment of the coal seam being drilled and evaluate the impact risk. This method is similar to the experimental method and device for coal stress measurement while drilling proposed in CN114017029B, and none of them can realize detection and identification of the properties of a rock mass. The near-bit measurement-while-drilling device and method for self-identifying multiple parameters underground coal mine proposed in CN115059449A is mainly used for large-size drilling equipment such as large-diameter pressure relief drilling machines underground coal mines, but is not suitable for equipment used in coal roadway roof support, such as jumbolters. The intelligent identification-while-drilling system and method for the lithology of the surrounding rock in coal mine roadways proposed in CN115077607A realizes intelligent identification of the properties of the surrounding rock with designed related experimental device. However, the device mainly has the following three shortcomings: 1. the drilling posture is horizontal, which is inconsistent with the construction posture of a real roof jumbolter and affects the identification accuracy of drilling parameters; 2. it can't apply a confining pressure, as a result, the conditions are quite different from the real conditions of the underground surrounding rock; 3. the drill bit of the drilling machine is inconsistent with a conventional twin-blade polycrystalline diamond compact (PDC) bit. Therefore, the method is unsatisfactory for lithology identification of the surrounding rock in coal roadways.

At present, the mainstream detection-while-drilling devices used in the coal mine field mainly have the following problems: 1. at present, the functions of the devices mainly focus on detection of the stress and gas in the surrounding rock, and there are few devices for detecting the properties of a rock mass while drilling; 2. the simulation target for drilling machine working condition of the drill bit of the experimental device is a large-diameter pressure relief drilling machine, which is inconsistent with an actual jumbolter in drilling dimension; 3. the drilling postures of existing detection-while-drilling experimental devices are mainly horizontal, which is inconsistent with the drilling posture of a jumbolter for coal roadways; 4. the existing experimental devices usually fix the experimental rock with linear constraints, and can't apply confining pressure, as a result, the conditions are quite different from the conditions of an underground rock mass in a stress field environment.

Therefore, it is necessary to design a detection-while-drilling device for composite rock stratum structures and an intelligent identification method to solve the above problems.

### SUMMARY OF THE INVENTION

The object of the present invention is to provide a detection-while-drilling device for composite rock stratum structures and an intelligent identification method, so as to solve the above problems, realize simulation and reproduction of a stress field environment under real conditions, and achieve a purpose of identifying and differentiating lithology by means of the response characteristics of different parameters during drilling of different rocks. To attain the object described above, the present invention employs the following technical solution: a detection-while-drilling device for composite rock stratum structures, comprising an equipment base, wherein a vertically-arranged supporting rod is fixedly connected to a middle part of a top end of the equipment base, a drilling hydraulic oil cylinder is fixedly connected to one side of the bottom of the supporting rod, a self-adaptive data acquisition-while-drilling module is fixedly connected to a piston end of the drilling hydraulic oil cylinder, the self-adaptive data acquisition-while-drilling module is slidably connected to the supporting rod, a sample table is arranged correspondingly above the self-adaptive data acquisition-while-drilling module, the sample table is fixedly connected to the supporting rod, a top limiting system is fixedly connected to a top end of the supporting rod, a rock gripper is arranged between the sample table and the top limiting system, the self-adaptive data acquisition-while-drilling module is electrically connected to the data acquisition device, and the data acquisition device is electrically connected to the data analysis terminal.

Preferably, the rock gripper comprises a base plate, a square through-hole is formed at the center of the base plate, universal rollers are arranged at four corners of a bottom end of the base plate respectively, the universal rollers are in rolling contact with a top end of the sample table, supporting hydraulic oil cylinders are fixedly connected to four corners of a top end of the base plate, piston ends of the supporting hydraulic oil cylinders pass through the base plate and are fixedly connected with supporting columns of rock sample clamping device, bottom ends of supporting columns of the rock sample clamping device abut against the top end of the sample table, the four supporting columns of rock sample clamping device are located between the four universal rollers, two fixed limiting steel plates that are parallel to each other and arranged vertically are fixedly connected to the top end of the base plate, the two fixed limiting steel plates are located between the four supporting hydraulic oil cylinders, and two steel plates of the hydraulic oil cylinders that are parallel to each other and arranged vertically are fixedly connected between the two fixed limiting steel plates; the top ends of the steel plates of the hydraulic oil cylinders and the fixed limiting steel plates are fixedly connected to a bottom end of a top plate together, and a top end of the top plate abuts against the bottom end of the top limiting system.

Preferably, the self-adaptive data acquisition-while-drilling module comprise a fixed plate, a bottom end of the fixed plate is fixedly connected to the piston ends of the drilling hydraulic oil cylinders, one side of the fixed plate is fixedly connected with a drilling guide rail groove, the drilling guide rail groove is slidably connected to the supporting rod, the bottom of the other side of the fixed plate is fixedly connected with a rotation speed acquisition unit, an output end of the rotation speed acquisition unit is fixedly connected to one end of a torque acquisition unit, the other end of the torque acquisition unit is fixedly connected with a drilling pipe groove, an axial force acquisition unit is arranged between the drilling pipe groove and the torque acquisition unit, and the axial force acquisition unit, the torque acquisition unit and the rotation speed acquisition unit are electrically connected to the data acquisition device.

Preferably, the sample table comprises a sample table flat surface, which is fixedly connected to the supporting rod, a drilling opening is formed at the center of the sample table flat surface, the universal rollers are in rolling contact with the sample table flat surface, bottom ends of supporting columns of the rock sample clamping device abut against the sample table flat surface, rock sample insertion inlets are arranged at two sides of the top end of the sample table flat surface, and a side wall of the sample table flat surface is fixedly connected with sample table guardrails.

Preferably, a side wall of the bottom of the supporting rod is fixedly connected with a guide rail, and the drilling guide rail groove is in sliding-fit with the guide rail.

An experimental method of the detection-while-drilling device for composite rock stratum structures, comprising the following steps:
S1. preparing rock samples having different strengths;
S2. testing basic parameters of the rock samples prepared in the step S1;
S3. placing a rock sample in the step S2 in the rock gripper and fixing it;
S4. controlling the self-adaptive data acquisition-while-drilling module to drill the rock sample, and monitoring parameter changes in the drilling process;
S5. removing the rock sample from the self-adaptive data acquisition-while-drilling module after the drilling is completed;
S6. repeating the steps S2-S5 to carry out experiments on the rock samples having different strengths and composites;
S7. repeating the steps S2-S5 and setting different confining pressures, to carry out experiments on the rock samples having different strengths;
S8. repeating the steps S2-S5 to carry out experiments on the rock samples having the same strength and different degrees of damage;
S9. establishing a database according to experimental data and carrying out analysis, and employing machine learning to train and learn with the database as an objective, so as to realize intelligent identification of roof structures.

Preferably, the rock samples having different strengths are prepared in the step S1 under a similarity criterion or on the basis of in-situ rocks.

Preferably, the basic parameters in the step S2 include uniaxial compressive strength, elastic modulus, internal friction angle and Poisson's ratio.

Preferably, the axial force acquisition unit, the rotation speed acquisition unit and the torque acquisition unit in the self-adaptive data acquisition-while-drilling module are positively correlated with the rock strength, and an initial mapping relation between the rock strength and the drilling parameters is shown in an expression; the mapping relation is incorporated into a machine learning algorithm, so as to realize intelligent identification of rock types; in the expression (1), E represents the rock strength, i represents data volume, k is an empirical coefficient, N is the rotation speed, M is the torque, F is the drilling force, a is an empirical coefficient of the drilling force, and b is a constant.

$$E = {\sum }_{i = 1}^{i} ( k \frac{{\overline{N}}_{i} \times {aF}_{i}}{\overline{M}} ) + b \qquad ( 1 )$$

Compared with the prior art, the present invention has the following advantages and technical effects:
(1) The present invention can simulate the drill bit size and drilling posture of a real jumbolter in a coal roadway, and can apply a confining pressure on the rock sample, thereby can realistically simulate the stress state of the surrounding rock;
(2) Through designing the torque, rotation speed, drilling rate, driving force and confining pressure in different grades, the response regularities of rock masses having different strengths, different degrees of damage and different quantities of fractures during drilling can be realized;
(3) Based on the drilling data, a mapping relationship between rock masses having different strengths, different degrees of damage and different quantities of fractures and different drilling parameters can be obtained through inversion, and finally the mapping relation can be used for dynamic identification of the properties of the rock mass;
(4) Based on the self-adaptive data acquisition-while-drilling module, through rapid improvement of a coalmine underground jumbolter, field application of detection while drilling for composite rock stratum structure can be realized quickly.

### BRIEF DESCRIPTION OF DRAWINGS

To explain the technical solution in the examples of the present invention or in the prior art more clearly, the drawings to be used in the examples will be introduced below briefly. Obviously, the drawings used in the description below only illustrate some examples of the present invention, and those having ordinary skills in the art can obtain other drawings based on these drawings without expending any creative labor.

FIG. 1 is a schematic structural diagram of the experimental device according to the present invention;

FIG. 2 is a schematic structural diagram of the rock gripper according to the present invention;

FIG. 3 is a schematic structural diagram of the self-adaptive data acquisition-while-drilling module according to the present invention; and

FIG. 4 is a schematic structural diagram of the sample table according to the present invention.

Wherein, in the figures: 1—sample table; 11—rock sample insertion inlet; 12—drilling opening; 13—sample table guardrail; 14—sample table flat surface; 2—rock gripper; 21—rock sample; 22—universal roller; 23—supporting column of rock sample clamping device; 24—steel plate of hydraulic oil cylinder; 25—fixed limiting steel plate; 26—supporting hydraulic oil cylinder; 3—top limiting system; 4—drilling hydraulic oil cylinder; 5—self-adaptive data acquisition-while-drilling module; 51—drilling pipe groove; 52—axial force acquisition unit; 53—torque acquisition unit; 54—rotation speed acquisition unit; 55—drilling guide rail groove; 6—displacement sensor; 7—guide rail; 8—equipment base.

### EMBODIMENTS

The technical solution in the examples of the present invention will be detailed below clearly and completely with reference to the accompanying drawings of the examples of the present invention. Obviously, the examples described herein are only some examples of the present invention, but not all possible examples of the present invention. All other examples, which can be obtained by those having ordinary skills in the art on the basis of the examples described herein without expending any creative labor, shall be deemed as falling in the scope of protection of the present invention.

The present invention will be further detailed in embodiments with reference to the accompanying drawings, in order to make the above-mentioned objects, features, and advantages of the present invention more easily understandable and more obvious.

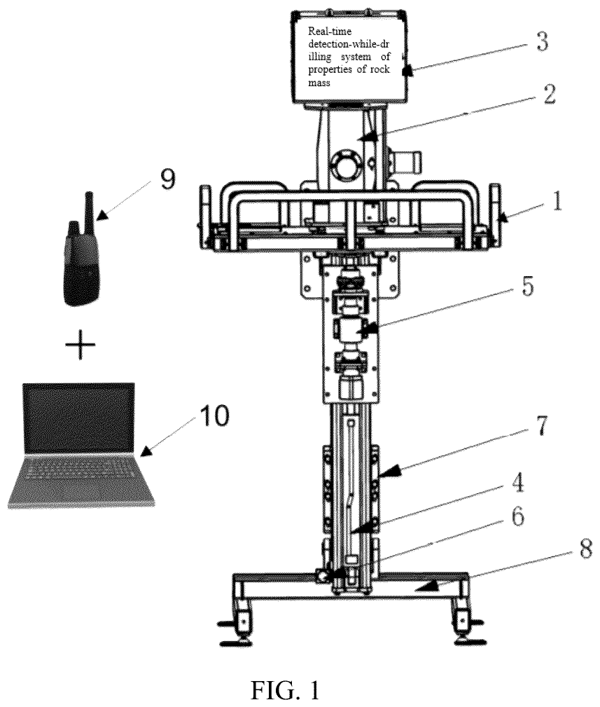

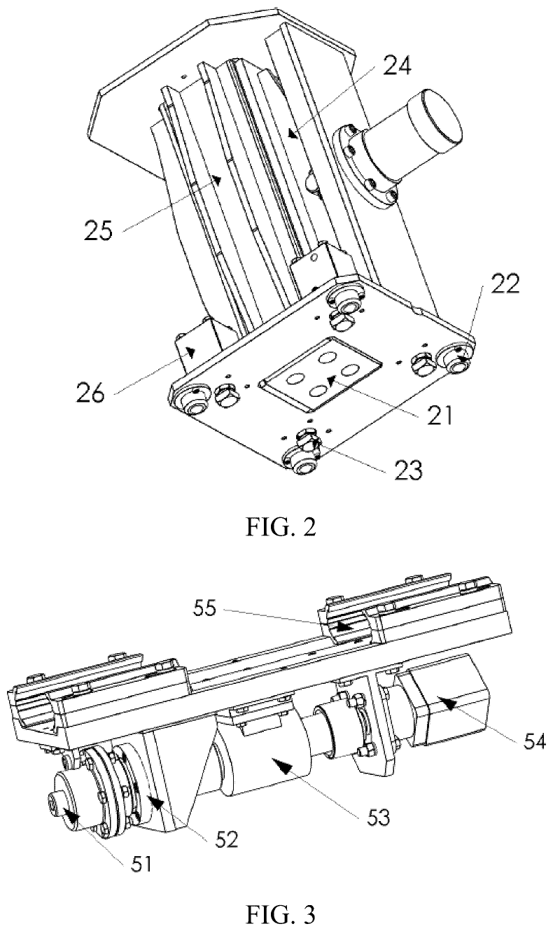

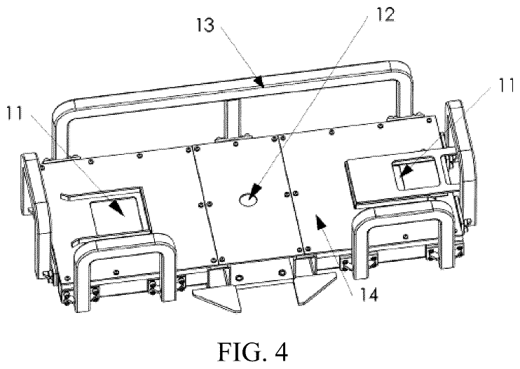

As shown in FIGS. 1 to 4, the present invention provides a detection-while-drilling device for composite rock stratum structures, which comprises an equipment base 8, wherein a vertically-arranged supporting rod is fixedly connected to a middle part of a top end of the equipment base 8, a drilling hydraulic oil cylinder 4 is fixedly connected to one side of the bottom of the supporting rod, a self-adaptive data acquisition-while-drilling module 5 is fixedly connected to a piston end of the drilling hydraulic oil cylinder 4, the self-adaptive data acquisition-while-drilling module 5 is slidably connected to the supporting rod, a sample table 1 is arranged correspondingly above the self-adaptive data acquisition-while-drilling module 5, the sample table 1 is fixedly connected to the supporting rod, a top limiting system 3 is fixedly connected to a top end of the supporting rod, a rock gripper 2 is arranged between the sample table 1 and the top limiting system 3, the self-adaptive data acquisition-while-drilling module 5 is electrically connected to the data acquisition device 9, and the data acquisition device 9 is electrically connected to the data analysis terminal 10.

Furthermore, a displacement sensor 6 is fixedly connected to the bottom end of the supporting rod for monitoring a displacement value.

The data acquisition device 9 can realize efficient wireless transmission of while-drilling data with Bluetooth and Wireless Fidelity techniques, thereby solving the problem of complicated data transmission in the underground environment of a coal mine, and the data analysis terminal 10 can realize intelligent analysis of the while-drilling data and identification of the rock strata.

In a further optimized solution, the rock gripper 2 comprises a base plate, a square through-hole is formed at the center of the base plate, universal rollers 22 are arranged at four corners of bottom end of the base plate respectively, the universal rollers 22 are in rolling contact with a top end of the sample table 1, supporting hydraulic oil cylinders 26 are respectively fixedly connected to four corners of top end of the base plate, piston ends of the supporting hydraulic oil cylinders 26 pass through the base plate and are fixedly connected with supporting columns 23 of rock sample clamping device, bottom ends of the supporting columns 23 of the rock sample clamping device abut against the top end of the sample table 1, the four supporting columns 23 of rock sample clamping device are located between the four universal rollers 22, two fixed limiting steel plates 25 that are parallel to each other and arranged vertically are fixedly connected to the top end of the base plate, the two fixed limiting steel plates 25 are located between the four supporting hydraulic oil cylinders 26, and two steel plates 24 of the hydraulic oil cylinders, which are parallel to each other and arranged vertically, are fixedly connected between the two fixed limiting steel plates 25; the top ends of the steel plates 24 of the hydraulic oil cylinders and the fixed limiting steel plates 25 are fixedly connected to a bottom end of a top plate together, and a top end of the top plate abuts against the bottom end of the top limiting system 3.

The hydraulic oil cylinder steel plates 24 and the fixed limiting steel plates 25 are used to realize application of a confining pressure on the rock sample 21; the rock gripper 2 can be moved freely on the sample table 1 by means of the universal rollers 22; and the rock gripper 2 is fixed between the top limiting system 3 and the sample table 1 by means of the supporting hydraulic oil cylinders 26.

The dimensions of the rock that can be clamped by the rock gripper 2 are within a range of 50 to 200 mm length, 50 to 200 mm width and 100 to 600 mm height.

In a further optimized solution, the self-adaptive data acquisition-while-drilling module 5 comprise a fixed plate, a bottom end of the fixed plate is fixedly connected to the piston ends of the drilling hydraulic oil cylinders 4, one side of the fixed plate is fixedly connected with a drilling guide rail groove 55, the drilling guide rail groove 55 is slidably connected to the supporting rod, the bottom of the other side of the fixed plate is fixedly connected with a rotation speed acquisition unit 54, an output end of the rotation speed acquisition unit 54 is fixedly connected to one end of a torque acquisition unit 53, the other end of the torque acquisition unit 53 is fixedly connected with a drilling pipe groove 51, an axial force acquisition unit 52 is arranged between the drilling pipe groove 51 and the torque acquisition unit 53, and the axial force acquisition unit 52, the torque acquisition unit 53 and the rotation speed acquisition unit 54 are electrically connected to the data acquisition device 9.

The self-adaptive data acquisition-while-drilling module 5 is moved on the supporting rod as a whole, resulting in a displacement; the rotating speed of the drilling pipe in the drilling pipe groove 51 is provided by the rotation speed acquisition unit 54; finally, the data change is monitored by means of the axial force acquisition unit 52 and the torque acquisition unit 53.

The drilling power part of the self-adaptive data acquisition-while-drilling module 5 can realize drilling with mining B19, B22 or any conventional drill pipe (with a diameter equal to or greater than 22 mm) and related bits; therefore, the self-adaptive data acquisition-while-drilling module is widely used.

In a further optimized solution, the sample table 1 comprises a sample table flat surface 14, which is fixedly connected to the supporting rod, a drilling opening 12 is formed at the center of the sample table flat surface 14, the universal rollers 22 are in rolling contact with the sample table flat surface 14, bottom ends of the supporting columns 23 of rock sample clamping device abut against the top end of the sample table flat surface 14, rock sample insertion inlets 11 are arranged at two sides of top end of the sample table flat surface 14, and a side wall of the sample table flat surface 14 is fixedly connected with sample table guardrails 13.

The rock gripper 2 is moved from the middle part of the sample table 1 to a position above the rock sample insertion inlet 11 on either side by means of the universal rollers 22, a rock sample 21 is placed in the rock gripper 2 via the rock sample insertion inlet 11 and then moved to a position above the drilling opening 12 by means of the universal rollers 22 to make preparation for the next step of experiment. The sample table guardrails 13 ensure that the rock gripper 2 is moved safely on the sample table flat surface 14.

In a further optimized solution, a side wall of the bottom of the supporting rod is fixedly connected with a guide rail 7, and the drilling guide rail groove 55 is in sliding-fit with the guide rail 7.

An experimental method of the detection-while-drilling device for composite rock stratum structures, comprising the following steps:
S1. preparing rock samples 21 having different strengths, wherein, the rock samples 21 may be cast at different cement-sand ratios in a laboratory, or may be drilled on the basis of typical composite roofs in the field of a coal mine.
S2. testing basic parameters of the rock samples 21 prepared in the step S1;
S3. placing a rock sample 21 in the step S2 in the rock gripper 2 and fixing it;

A rock sample 21 is placed in a sample groove through the rock sample insertion inlet 11 on either side of the sample table 1, a confining pressure is applied on the rock sample 21 in directions X and Y by means of the rock gripper 2, the rock gripper 2 is moved to the middle part of the sample table 1, and a control device is operated to actuate the supporting columns 23 of rock sample clamping device, so that the rock gripper 2 leaves the sample table 1 under the action of the universal rollers 22.
S4. controlling the self-adaptive data acquisition-while-drilling module 5 to drill the rock sample 21, and monitoring parameter changes in the drilling process, such as torque and axial force, etc.;

A drill pipe and a drill bit are mounted on the drilling pipe groove 51, an electro-hydraulic servo control system is used to activate the rotation speed acquisition unit 54 and propel displacement and control the real-time parameters, so that the drill bit starts drilling operation, and the parameters are acquired in the drilling process with torque, axial force, displacement and rotation speed sensors; as the drilling machine moves forward, the drilling hydraulic oil cylinder 4 moves in the Z direction, so that the self-adaptive data acquisition-while-drilling module 5 is moved as a whole, and an initial driving force is given for the drill bit; in the drilling part, a rated rotation speed is set for the drilling spindle with the rotation speed acquisition unit 54, and the real-time change of drilling process parameters is monitored with the speed sensor and the torque acquisition unit 53.
S5. removing the rock sample 21 from the self-adaptive data acquisition-while-drilling module 5 after the drilling is completed;

When drilling is finished, the displacement of the self-adaptive data acquisition-while-drilling module 5 is stopped, and the self-adaptive data acquisition-while-drilling module 5 is moved back by controlling the displacement, so as that the drill bit is withdrawn from the rock sample 21.
S6. repeating the steps S2 to S5 to carry out experiments on the rock samples 21 having different strengths and composites;

Rock samples 21 having different strengths and composites are configured, the steps S2 to S5 are repeated with the experimental device, the working parameters for the rock samples 21 having different strengths and composites during drilling, including torque, axial force, displacement and rotation speed, are logged, the response characteristics of the rock masses having different strengths and composites to main drilling parameters during drilling are studied, and a mapping relationship between them is established, so as to facilitate the detection of rock mass structures while drilling.
S7. repeating the steps S2 to S5 and setting different confining pressures, to carry out experiments on the rock samples 21 having different strengths;

The confining pressure in different gradients is changed, rock samples 21 having different strengths are used, and the steps S2 to S5 are repeated, to simulate and study the drilling response characteristics of rock samples 21 having different strengths under the impact of the confining pressure, and study the influence relationship between the stress environment of the surrounding rock and rock strength identification, so as to provide reference for identification of the properties of the surrounding rocks at different depths.
S8. repeating the steps S2 to S5 to carry out experiments on the rock samples 21 having the same strength and different degrees of damage;

Rock samples 21 having the same strength but different degrees of damage are used, or fractures having different structures and different quantities are produced in advance in rock samples 21, the steps S2 to S5 are repeated with the experimental device, and the response regularities of rock samples 21 having different degrees of damage to different drilling parameters are logged, to simulate and study detection and identification of damage degrees/fractures of rock mass while drilling.
S9. establishing a database according to experimental data and carrying out analysis, and employing machine learning to train and learn with the database as an objective, so as to realize intelligent identification of roof structures.

A corresponding database is established for the obtained massive experimental drilling data, structural processing and analysis are carried out according to the data statistics; with the database as an objective, artificial intelligence training and learning are carried out with artificial intelligence means such as machine learning, and finally intelligent identification of the roof structure is realized.

In the present invention, a regression algorithm of end-to-end regression based on a deep learning model is employed. The algorithm uses continuous value data and various date sources such as geological images for training, so as to realize high-precision prediction of the roof structure.

The specific implementation method is as follows:

Data preparation: continuous value data is acquired while drilling, and, in conjunction with data sources such as geological images, a training data set and a test data set are generated.

Model design: a convolutional neural network (CNN) and a long short-term memory network (LSTM) are used in combination to construct an end-to-end regression model. The input layer receives continuous value data and a variety of data sources such as geological images, and finally outputs a prediction result after a series of processing including convolution, pooling and LSTM.

Model training: the model is trained with the training data set, and the effect of the model is verified with the testing data set. In the training process, data enhancement and regularization and other techniques can be used to improve the robustness and generalization ability of the model.

Model optimization: the performance of the model is further optimized by means of model structure adjustment, hyperparameter optimization, ensemble learning and other methods, to improve the prediction accuracy and stability of the model.

Model application: the trained model is deployed for actual detection while drilling to predict the roof structure. Adjustment and optimization are performed according to the prediction result, and the algorithm and model are iteratively optimized.

The specific structure of the combination of a convolutional neural network and a long short-term memory network is as follows:

Input layer: it accepts continuous value data and a variety of data sources such as geological images.

Convolution layer 1: it uses a 3×3 convolution kernel to extracts the features of the bottom layer and output features map of 64 channels.

Maximum pooling layer 1: it performs a maximum pooling operation on the output of the convolution layer 1 to reduce the size of the feature map.

Convolution layer 2: it uses a 3×3 convolution kernel to further extract the features of the intermediate layer and outputs feature maps of 128 channels.

Maximum pooling layer 2: it performs a maximum pooling operation on the output of the convolution layer 2.

LSTM layer 1: it uses LSTM layers of 128 units to process the output of the pooling layer 2 while preserving the time series information of the features of the intermediate layer.

Dropout layer: it performs a random deactivation operation on the output of the LSTM layer 1 in order to prevent over-fitting.

LSTM layer 2: it further uses the LSTM layers of 128 units to process the output of the Dropout layer and further fuse the time series information.

Fully connected layer 1: after the output of the LSTM layer 2 is flattened, the fully connected layer 1 performs dimension transformation and feature mapping, and outputs a 256-dimension feature vector.

Fully connected layer 2: the fully connected layer is used again to map the feature vector to a final prediction result space and output continuous values to realize a result of detection of the roof structure while drilling.

In a further optimized solution, the rock samples 21 having different strengths are prepared in the step S1 under a similarity criterion or on the basis of in-situ rocks.

The similarity criterion is as follows: the uniaxial compressive strengths of different types of rocks is simulated by casting concrete rock materials, so as to achieve a similarity in uniaxial compressive strength between the cast samples and real rocks. Usually, uniaxial compressive tests are carried out on samples with a size of 100*100*100 mm, which are cast at different ratios of water, cement and river sand, and the compressive strengths are measured. If the error of uniaxial compressive strength between the sample rock and real rock is smaller than 10%, it is considered that the sample meet the similarity criterion.

In a further optimized solution, the basic parameters in the step S2 include uniaxial compressive strength, elastic modulus, internal friction angle and Poisson's ratio.

Basic parameters (such as uniaxial compressive strength, elastic modulus, internal friction angle and Poisson's ratio, etc.) of the rock samples 21 having different strengths are measured with laboratory basic mechanical tests.

In a further optimized solution, the axial force acquisition unit 52, the rotation speed acquisition unit 54 and the torque acquisition unit 53 in the self-adaptive data acquisition-while-drilling module 5 are positively correlated with the rock strength, and an initial mapping relation between the rock strength and the drilling parameters is shown in an expression 1; the mapping relation is incorporated into a machine learning algorithm, so as to realize intelligent identification of rock types; in the expression 1, E represents the rock strength, i represents a data volume, k is an empirical coefficient, N is the rotation speed, M is the torque, F is the drilling force, a is an empirical coefficient of the drilling force, and b is a constant.

$$E = {\sum }_{i = 1}^{i} ( k \frac{{\overline{N}}_{i} \times {aF}_{i}}{\overline{M}} ) + b \qquad ( 1 )$$

In the description of the present invention, it should be understood that the orientational or positional relations indicated by terms “longitudinal”, “transverse”, “above”, “below”, “front”, “rear”, “left”, “right”, “vertical”, “horizontal”, “top”, “bottom”, “inside” or “outside”, etc., are based on the orientational or positional relations indicated in the accompanying drawings. They are used only to ease the description of the present invention, rather than indicating or implying that the involved device or component must have a specific orientation or must be constructed and operated in a specific orientation. Therefore, the use of these terms shall not be deemed as constituting any limitation on the present invention.

1. A detection-while-drilling device for composite rock stratum structures, comprising a data analysis terminal, a data acquisition device and an equipment base, wherein a vertically-arranged supporting rod is fixedly connected to a middle part of a top end of the equipment base, a drilling hydraulic oil cylinder is fixedly connected to one side of the bottom of the supporting rod, a self-adaptive data acquisition-while-drilling module is fixedly connected to a piston end of the drilling hydraulic oil cylinder, the self-adaptive data acquisition-while-drilling module is slidably connected to the supporting rod, a sample table is arranged correspondingly above the self-adaptive data acquisition-while-drilling module, the sample table is fixedly connected to the supporting rod, a top limiting system is fixedly connected to a top end of the supporting rod, a rock gripper is arranged between the sample table and the top limiting system, the self-adaptive data acquisition-while-drilling module is electrically connected to the data acquisition device, and the data acquisition device is electrically connected to the data analysis terminal.

2. The detection-while-drilling device for composite rock stratum structures according to claim 1, wherein the rock gripper comprises a base plate, a square through-hole is formed at the center of the base plate, universal rollers are arranged at four corners of a bottom end of the base plate respectively, the universal rollers are in rolling contact with a top end of the sample table, supporting hydraulic oil cylinders are fixedly connected to four corners of the top end of the base plate, piston ends of the supporting hydraulic oil cylinders pass through the base plate and are fixedly connected with supporting columns of rock sample clamping device, bottom ends of the supporting columns of the rock sample clamping device abut against the top end of the sample table, the four supporting columns of the rock sample clamping device are located between the four universal rollers, two fixed limiting steel plates that are parallel to each other and arranged vertically are fixedly connected to the top end of the base plate, the two fixed limiting steel plates are located between the four supporting hydraulic oil cylinders, and two steel plates of the hydraulic oil cylinders that are parallel to each other and arranged vertically are fixedly connected between the two fixed limiting steel plates; the top ends of the steel plates of the hydraulic oil cylinders and the fixed limiting steel plates are fixedly connected to a bottom end of a top plate together, and a top end of the top plate abuts against the bottom end of the top limiting system.

3. The detection-while-drilling device for composite rock stratum structures according to claim 2, wherein the self-adaptive data acquisition-while-drilling module comprise a fixed plate, a bottom end of the fixed plate is fixedly connected to the piston ends of the drilling hydraulic oil cylinders, one side of the fixed plate is fixedly connected with a drilling guide rail groove, the drilling guide rail groove is slidably connected to the supporting rod, the bottom of the other side of the fixed plate is fixedly connected with a rotation speed acquisition unit, an output end of the rotation speed acquisition unit is fixedly connected to one end of a torque acquisition unit, the other end of the torque acquisition unit is fixedly connected with a drilling pipe groove, an axial force acquisition unit is arranged between the drilling pipe groove and the torque acquisition unit, and the axial force acquisition unit, the torque acquisition unit and the rotation speed acquisition unit are electrically connected to the data acquisition device.

4. The detection-while-drilling device for composite rock stratum structures according to claim 3, wherein the sample table comprises a sample table flat surface, which is fixedly connected to the supporting rod, a drilling opening is formed at the center of the sample table flat surface, the universal rollers are in rolling contact with the sample table flat surface, bottom ends of the supporting columns of the rock sample clamping device abut against the top end of the sample table flat surface, rock sample insertion inlets are arranged at two sides of a top end of the sample table flat surface, and a side wall of the sample table flat surface is fixedly connected with sample table guardrails.

5. The detection-while-drilling device for composite rock stratum structures according to claim 3, wherein a side wall of a bottom of the supporting rod is fixedly connected with a guide rail, and the drilling guide rail groove is in sliding-fit with the guide rail.

6. An intelligent identification method of the detection-while-drilling device for composite rock stratum structures according to claim 1, comprising the following steps:
S1) preparing rock samples having different strengths;
S2) testing basic parameters of the rock samples prepared in the step S1;
S3) placing a rock sample obtained in the step S2 in the rock gripper and fixing it;
S4) controlling the self-adaptive data acquisition-while-drilling module to drill the rock sample, and monitoring parameter changes in the drilling process;
S5) removing the rock sample from the self-adaptive data acquisition-while-drilling module after the drilling is completed;
S6) repeating the steps S2 to S5 to carry out experiments on the rock samples having different strengths and composites;
S7) repeating the steps S2 to S5 and setting different confining pressures, to carry out experiments on the rock samples having different strengths;
S8) repeating the steps S2 to S5 to carry out experiments on the rock samples having the same strength and different degrees of damage; and
S9) establishing a database according to experimental data and carrying out analysis, and employing machine learning to train and learn with the database as an objective, so as to realize intelligent identification of roof structures.

7. The intelligent identification method of the detection-while-drilling device for the composite rock stratum structure according to claim 6, wherein the rock samples having different strengths are prepared in the step S1 under a similarity criterion or on the basis of in-situ rocks.

8. The intelligent identification method of the detection-while-drilling device for the composite rock stratum structure according to claim 6, wherein the basic parameters in the step S2 include uniaxial compressive strength, elastic modulus, internal friction angle and Poisson's ratio.

9. The intelligent identification method of the detection-while-drilling device for the composite rock stratum structure according to claim 6, characterized in that, wherein the axial force acquisition unit, the rotation speed acquisition unit and the torque acquisition unit in the self-adaptive data acquisition-while-drilling module are positively correlated with the rock strength, and an initial mapping relation between the rock strength and the drilling parameters is shown in an expression; the mapping relation is incorporated into a machine learning algorithm, so as to realize intelligent identification of rock types; in the expression, E represents the rock strength, i represents a data volume, k is an empirical coefficient, N is the rotation speed, M is the torque, F is the drilling force, a is an empirical coefficient of the drilling force, and b is a constant,
$$E = {\sum }_{i = 1}^{i} ( k \frac{{\overline{N}}_{i} \times {aF}_{i}}{\overline{M}} ) + b . \qquad ( 1 )$$
